(19) United States
(12) Patent Application Publication
(10) Pub. No.: US 2020/0179084 A1
(43) Pub. Date: Jun. 11, 2020

(54) BIORESORBABLE METAL ALLOY MEMBRANES, METHODS OF MAKING, AND METHODS OF USE

(71) Applicant: University of Florida Research Foundation, Inc., Gainesville, FL (US)

(72) Inventors: Michele Viola MANUEL, Gainesville, FL (US); Ikramuddin AUKHIL, Gainesville, FL (US)

(21) Appl. No.: 16/638,939

(22) PCT Filed: Aug. 14, 2018
(86) PCT No.: PCT/US18/46695

§ 371 Date: Feb. 13, 2020

Related U.S. Application Data
(60) Provisional application No. 62/544,941, filed on Aug. 14, 2017.

(51) Int. Cl. A61C 8/00; A61C 8/02; A61K 6/17

(52) U.S. Cl. CPC A61C 8/0012; A61K 6/17; A61C 8/0006

(57) ABSTRACT

Embodiments of the present disclosure provide for structures including bioresorbable alloy membrane (e.g., Mg-, Fe-, Zn-based alloy membranes that include calcium, strontium, and/or manganese), methods of guided bone regeneration, and the like. In an aspect, the membrane can be a periodontal mesh that is biodegradable, bioerodible, and biocompatible and has a life time (e.g., 1-4 months) in line with what is desired for such procedures.

### CLAIM OF PRIORITY TO RELATED APPLICATION

This application claims priority to co-pending U.S. provisional application entitled “BIORESORBABLE METAL ALLOY MEMBRANES, METHODS OF MAKING, AND METHODS OF USE” having Ser. No. 62/544,941, filed on Aug. 14, 2017, which is entirely incorporated herein by reference.

### BACKGROUND

Biomaterials are used in numerous medical applications today, such as fixation devices, replacements and surgical equipment. Implants are typical examples of a biomaterial application and there are several different implant materials used today. Many of these are however designed to stay in the body permanently even though they only serve their function temporarily. Even if the materials are biocompatible there are several complications associated with long term presence of implants.

### SUMMARY

Embodiments of the present disclosure provide for structures including bioresorbable alloy membrane (e.g., Mg-, Fe-, Zn-based alloy membranes that include calcium, strontium, and/or manganese), methods of guided bone regeneration, and the like. In an aspect, the membrane can be a periodontal mesh that is biodegradable, bioerodible, and biocompatible and has a life time (e.g., 1-4 months) in line will) what is desired for such procedures.

In an aspect, the present disclosure provides for a structure comprising: a bioresorbable alloy membrane having a plurality of pores, wherein the alloy membrane has a thickness of about 0.1 to 0.5 mm, wherein the pores have a cross-sectional dimension of about 0.1 to 2 mm, wherein the alloy membrane is one or more of biodegradable, bioerodible, and biocompatible. The alloy membrane can be a Fe-based alloy membrane, a Zn-based alloy membrane, or an Mg-based alloy membrane. The term “X-based alloy” means that the alloy can include other components in the alloy such as calcium, strontium, and/or manganese in addition to “X” (Fe, Zn, and/or Mg).

In an aspect, the method for guided bone regeneration, comprising: disposing a bone paste into an area for which bone is to be formed; and disposing a bioresorbable alloy membrane as described herein around the bone paste to contain the bone paste in the area.

In an aspect, the structure comprising: a periodontic bioresorbable alloy mesh having a plurality of pores, wherein the alloy membrane has a thickness of about 0.25 to 0.3 mm, wherein the pores have a cross-sectional dimension of about 0.1 to 2 mm, wherein the alloy membrane is one or more of biodegradable, bioerodible, and biocompatible.

Other structures, compositions, methods, features, and advantages will be, or become, apparent to one with skill in the art upon examination of the following drawings and detailed description. It is intended that all such additional structures, systems, methods, features, and advantages be included within this description, be within the scope of the present disclosure, and be protected by the accompanying claims.

### BRIEF DESCRIPTION OF THE DRAWINGS

Many aspects of this disclosure can be better understood with reference to the following drawings.

FIG. 1 illustrates the evolution of hydrogen gas in a magnesium-based implant screw implant located in the femur of 3 different goats.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of chemistry, material science, biology, dentistry, and the like, which are within the skill of the art. Such techniques are explained fully in the literature.

Prior to describing the various embodiments, the following definitions are provided and should be used unless otherwise indicated.

Unless otherwise defined, all technical and scientific terms used herein have the same meaning as commonly understood by one of ordinary skill in the art of chemistry, physics, fluid dynamics, and the like. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

The term “biodegradable” includes that all or parts of the material will degrade over time by the action of enzymes, by hydrolytic action and/or by other similar mechanisms in the oral cavity. In various embodiments, “biodegradable” includes that the material can break down or degrade within the oral cavity to non-toxic components.

The term “bioerodible” means that the material or portion thereof will erode or degrade over time due, at least in part, to contact with substances found in the surrounding tissue, fluids or by cellular action.

The term “bioresorbable” means that the material or portion thereof will be broken down and resorbed within the human body, for example, by a cell or tissue.

The term “biocompatible” means that the material will not cause substantial tissue irritation or necrosis at the target tissue site.

### Discussion

Embodiments of the present disclosure provide for structures including bioresorbable alloy membrane (e.g., Mg-, Fe-, Zn-based alloy membranes that include calcium, strontium, and/or manganese), methods of guided bone regeneration, and the like. In an embodiment the structure can be used in guided bone regeneration such as in periodontal and craniofacial applications, where in an aspect the membrane is a mesh that can maintain a negative space and promote and/or support bone regeneration, specifically bone regeneration in the negative space (which may include bone paste). After a period of time (e.g., a few months (about 1-3 months)) the mesh is reabsorbed into the body.

In general, membranes (also referred to as “alloy membranes”) of the present disclosure can protect the periodontal defect (e.g., tooth abstraction) from saliva, other liquids, food, bacteria and/or other material that slows healing of the periodontal defect. In some embodiments, the membrane provided reduce the number of surgical procedures. In some embodiments, the membrane allow guided tissue regeneration where, the cementum, alveolar bone and periodontal ligament producing cells have the ability to become established on the tooth root surface by isolating the periodontal defect from unwanted saliva, other liquids, food, bacteria and/or other material that slows healing of the periodontal defect. The membrane provided allow proper healing of the periodontal defect and, in some embodiments, gingival tissue can be attached to it so that a dental implant can be affixed, for example.

In an aspect, the membrane can be a thin mesh barrier placed around or in the area or space and retrained (e.g., sutured) in place. Without such a barrier, fluids easily access the area while the bone attempts to repair itself. In an aspect, a bone paste or similar material (as known in the art) can be disposed within all or a portion of the negative space. The bone can grow into the mesh material and the mesh material can degrade and be resorbed within the human body. In this way the membrane is biodegradable, bioerodible, bioresorbable, and biocompatible.

Embodiments of the present disclosure can be advantageous in that the membrane provides structural rigidity during placement and for a time frame to form the bone (e.g., maintain a negative space for the bone to form). In addition, the membrane promotes osteointegration with minimal or no detrimental immune response. Furthermore, the membrane is bioabsorbable, unlike titanium membrane, eliminating a secondary procedure to remove the membrane, where such secondary procedures are necessary and often problematic for titanium membranes. Thus in situations where the membrane is used in treatments of periodontitis or alveolar ridge reconstruction, the risk and patient experience can be improved while also greatly reducing the cost of treatment and patient morbidity.

Magnesium, iron, and zinc based alloys are excellent implant material due to their attractive mechanical properties and non-toxicity. It has a high corrosion rate, especially in chloride containing solutions, which means that it will degrade in the human body. The degradation rate can be controlled for alloy membranes of the present disclosure which allows for the alloy membranes to be used in guided bone regeneration procedures, in particular guided bone regeneration procedures in periodontal applications.

Embodiments of the present disclosure provide for a degradable material by selecting the alloying elements for purposes of obtaining optimal mechanical functionality while maintaining biocompatibility. Calcium is an essential element for the human body and is non-toxic. Strontium is present in human bones and has been shown to promote osteoblast function and increase bone formation when added to hydroxyapatite, as compared to pure hydroxapatite. Manganese improves the mechanical properties of iron based alloys while being an essential element for the human body and is non-toxic. This creates the opportunity to develop membranes that can completely dissolve within the body and that release dissolution products that are 100% biocompatible and enhance the biological processes in bone. Use of magnesium-based alloy membranes containing calcium and strontium greatly reduces the risk of potential toxicity by the degradation products being released from the membranes.

In addition to their biological response, calcium and strontium are known to strengthen magnesium alloys while increasing their corrosion resistance. Controlling these elements and the corresponding microstructures that develop upon processing, our magnesium-based alloy membrane can be designed with controllable degradation rates and mechanical properties (e.g., ductility).

In an embodiment, the membrane includes a plurality of pores. The pores extend through the membranes so that biological components can pass through the membranes. In an aspect, the membrane can structurally and dimensionally resemble a mesh, such as a titanium mesh, used in periodontal procedures, where rather than using titanium alloy of the present disclosure are used with similar dimensions. In an embodiment, the pores have a cross-sectional dimension to permit the passage of biological components to form bone, and the cross-sectional dimension (e.g., diameter, length, width, etc.) can be about 0.1 to 2 mm, 0.1 to 1 mm, about 0.2 to 2 mm, or about 0.2 to 1 mm. In an aspect, the pores extend through the thickness of the membrane and can alternatively be referred to as a channel through the membrane. In an embodiment, the cross-sectional area can be polygonal, non-polygonal, circular, or the like, where each pore cross-sectional area can be uniform through the alloy membrane or not uniform. In an aspect, the membrane can have a mesh construction. In an aspect, a mesh construction can be appear as a film having a plurality of pores therein or as a number of strand, bands, sections, etc. of material interconnected but having spaces (pores) within the material. In an aspect of the membrane of the present disclosure the pores can be made (e.g., drilled) into the membrane so that it has the appearance of mesh, specifically, the membrane can be made to look dimensionally similar to a titanium mesh with similar pores sizes and thickness. In an embodiment all of the pores can have the same cross-sectional shape and dimension or the alloy membranes pores can be two or more different cross-sectional shapes and/or dimensions. In an embodiment, the membrane can have a thickness of about 0.05 to 0.5 mm, about 0.05 to 0.3 mm, about 0.05 to 0.2 mm, about 0.05 to 0.15, or about 0.08 to 0.12.

Embodiments of the present disclosure are suitable for guided bone regeneration for at least the following reasons: high ductility and low degradation rate. In the thermomechanically-processed condition, it displays considerable ductility up to 20% in both tension and compression, near the maximum possible for Mg alloys at room temperature, for example. This is advantageous because during placement in a dental procedure, for example, the dental surgeon permanently bends the membrane to the precise shape of the desired ridge regrowth. Considerable plastic deformation is advantageous for this purpose and must be balanced against the thickness of samples, as the thicker a sheet, the higher percent deformation required for a certain radius of bending. In general, magnesium-based membranes have less ductility than comparable Ti alloys (Pure Ti exhibits elongation from 50%-100%), it may need to be thinner (e.g., less than 0.2 mm, less than about 0.18 mm, less than about 0.16 mm) in some applications to compensate for this effect (e.g., about 0.1 mm) instead of the typical thickness of 0.2 mm for Ti. The thickness of the implant is considered for this application, as it corresponds to a high surface/volume ratio and necessitates an alloy with as low a degradation rate as possible.

In the thermomechanically-worked condition, this alloy exhibits an in vitro degradation rate (in simulated body fluid) as low as any Mg alloy currently known. In an embodiment, the alloy membrane is degradable in biological condition such as those present during bone growth or regeneration. This low degradation rate limits the rate of hydrogen gas evolution, which, if high, could have negative effects on tissue growth in the healing periodontal and bone tissue. This phenomenon is not as dangerous in barrier membrane application as some others however, as the membrane is surrounded by soft tissue that can expand to accommodate unanticipated gas bubbles.

Strength requirements are relatively low for barrier membranes (compared to load bearing orthopedic screws or plates), but the alloy membrane displays sufficient structural integrity to hold soft tissues during bone mineralization. One particular benefit of the alloy membranes of the present disclosure is that all alloy components are osteogenic, promoting the formation of new bone through the release of Ca and Sr and the general alkalinization of the local environment.

In an embodiment, the alloy membrane can have a ductility so that the alloy membrane can be formed into the desired shape for the guided bone regeneration area. One of skill in the art can design the alloy membrane to have the ductility for the particular application.

In an embodiment, the alloy membrane can have a degradation rate that is long enough so that the bone can be formed and hard enough to not be deformed (e.g., 1 to 4 months or 1 to 3 months or 1 to 2 months, depending upon the application). One of skill in the art can design the alloy membrane to have the degradation rate for the particular application. In an embodiment, the alloy membrane can be Mg-, Fe-, Zn-based alloy membranes that includes calcium, strontium, and/or manganese (e.g., each independently about 0.3 to 10 weight percent of the alloy membrane), where the remainder is Mg, Fe, or Zn (or a combination of Mg, Fe, and/or Zn). In an aspect, the Mg-based alloy includes calcium and/or strontium. In another aspect, the Fe-based alloy includes manganese. In yet another aspect, the Zn-based alloy includes magnesium and/or iron. In an aspect, the Mg-, Fe-, Zn-based alloy membranes can optionally include one or more of scandium, yttrium, gadolinium, cerium, neodymium, dysprosium, or a combination thereof each one independently in amounts that can be about 0.01 to 5 percent weight, as these elements can be used to modify (e.g., lengthen) the degradation rate.

In an embodiment, the alloy membrane can include, by weight percentage, about 0.3 to 10 weight percent calcium; about 0.3 to 10 percent weight strontium; and about 50 to 99.5 weight percent magnesium. In an exemplary embodiment, the alloy comprises about 0.3 to 2 weight percent strontium or about 0.6 to 1 weight percent strontium. In an embodiment, the alloy can include about 0.6 to 2 weight percent of calcium or about 0.6 to 1 weight percent calcium. In an embodiment, the alloy can include about 0.6 to 2 weight percent of calcium, about 0.6 to 2 weight percent of strontium, and about 96 to 98.8 weigh percent of magnesium.

In an embodiment, the alloy membrane can include, by weight percentage, about 0.3 to 10 weight percent manganese and about 50 to 99.5 weight percent iron. In an exemplary embodiment, the alloy comprises about 0.3 to 2 weight percent manganese or about 0.6 to 1 weight percent manganese. In an embodiment, the alloy can include about 0.6 to 4 weight percent of manganese and about 96 to 99.4 weigh percent of iron.

In an embodiment, the alloy membrane can include, by weight percentage, about 0.3 to 10 weight percent iron; about 0.3 to 10 percent weight magnesium; and about 50 to 99.5 weight percent zinc. In an exemplary embodiment, the alloy comprises about 0.3 to 2 weight percent iron or about 0.6 to 1 weight percent iron. In an embodiment, the alloy can include about 0.6 to 2 weight percent of magnesium or about 0.6 to 1 weight percent magnesium. In an embodiment, the alloy can include about 0.6 to 2 weight percent of iron, about 0.6 to 2 weight percent of magnesium, and about 96 to 98.8 weigh percent of zinc.

According to certain embodiments, embodiments of the present disclosure relate to a bioresorbable, non-toxic, osteogenic magnesium-based alloy membrane. As used herein, the term osteogenic relates to the property of facilitating in growth of bone (osteoconductivity) and/or promoting new bone growth (osteoinductivity).

In alternative embodiment, embodiments of the present disclosure relate to an alloy membrane that comprises magnesium, calcium and strontium and which is substantially free from aluminum, manganese, and/or zirconium. As used herein, the term “substantially free” means that the element or compound comprises less than 3 percent by weight of the alloy, less than 1 percent by weight of the alloy, or less than 0.1 percent by weight of the alloy.

Alloy membranes of the present disclosure can be made using methods such as extruding processes and hot roller processes and the pores can be formed through drilling (e.g., mechanical drilling, laser drilling, etc.) or other techniques.

### EXAMPLE

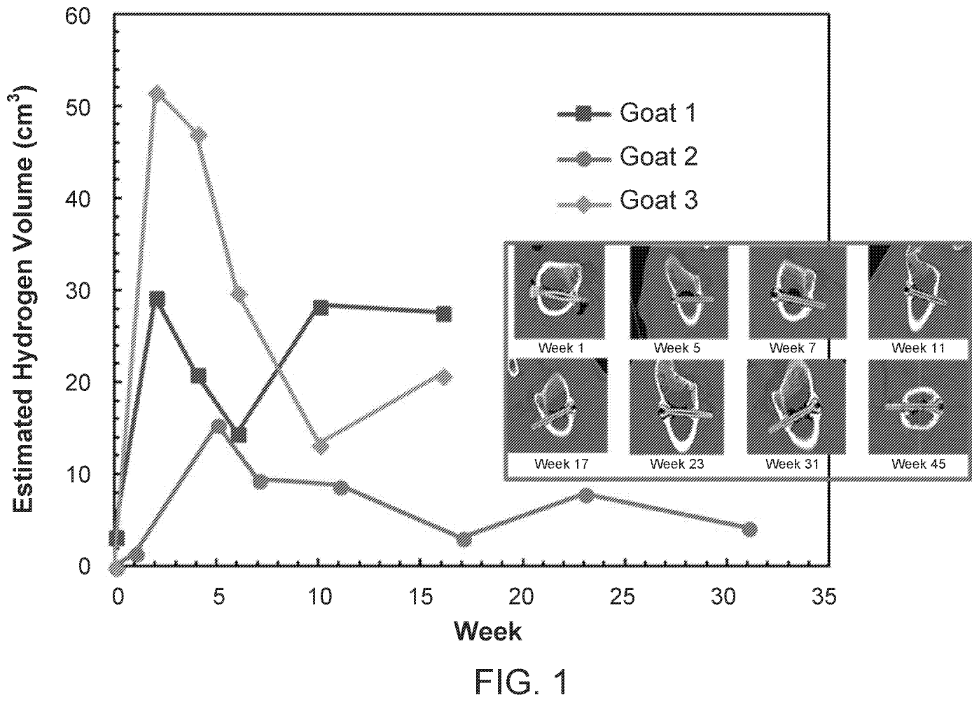

FIG. 1 illustrates the evolution of hydrogen gas in a magnesium-based implant screw implant located in the femur of 3 different goats. The data indicates an increase in gas evolution in the first few weeks with a reduction in later weeks as the implant is degrading. The inset X-ray images for Goat 2 of the screw during the analysis period are shown in the micrographs illustrating the slow degradation rate of the implant in a large animal.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

1. A structure comprising:
a bioresorbable alloy membrane having a plurality of pores, wherein the alloy membrane has a thickness of about 0.1 to 0.5 mm, wherein the pores have a cross-sectional dimension of about 0.1 to 2 mm, wherein the alloy membrane is one or more of biodegradable, bioerodible, and biocompatible.

2. The structure of claim 1, wherein the alloy membrane is a Fe-based alloy membrane, a Zn-based alloy membrane, or a Mg-based alloy membrane.

3. The structure of claim 2, wherein the alloy membrane comprises about 0.2 to 10 weight percent calcium, about 0.2 to 10 weight percent strontium, and about 80 to 99.6 weight percent magnesium.

4. The structure of claim 2, wherein the alloy membrane comprises about 0.3 to 2 weight percent calcium, about 0.3 to 2 weight percent strontium, and about 96 to 99.4 weight percent magnesium.

5. The structure of claim 2, wherein the alloy membrane comprises about 0.6 to 4 weight percent of manganese and about 96 to 99.4 weight percent of iron.

6. The structure of claim 2, wherein the alloy membrane comprises about 0.6 to 2 weight percent of iron, about 0.6 to 2 weight percent of magnesium, and about 96 to 98.8 weigh percent of zinc.

7. The structure of claim 1, wherein the alloy membrane is a bioresorbable, non-toxic, magnesium alloy.

8. The structure of claim 1, wherein the alloy membrane is a non-toxic, non-immunoreactive periodontic structure.

9. The structure of claim 1, wherein the alloy is substantially free from aluminum, manganese, zirconium, or a combination thereof.

10. The structure of claim 1, wherein the alloy includes one or more of scandium, yttrium, gadolinium, cerium neodymium, dysprosium, or a combination thereof each one independently in amounts that can be about 0.01 to 5 percent weight.

11. A method for guided bone regeneration, comprising:
disposing a bone paste into an area for which bone is to be formed; and
disposing a bioresorbable alloy membrane of claim 1 around the bone paste to contain the bone paste in the area.

12. A structure comprising:
a periodontic bioresorbable alloy mesh having a plurality of pores, wherein the alloy membrane has a thickness of about 0.25 to 0.3 mm, wherein the pores have a cross-sectional dimension of about 0.1 to 2 mm, wherein the alloy membrane is one or more of biodegradable, bioerodible, and biocompatible.
